(19) United States
(12) Patent Application Publication
(10) Pub. No.: US 2022/0349016 A1
(43) Pub. Date: Nov. 3, 2022

(54) MOLECULAR MARKERS FOR IDENTIFICATION OF DISEASE RESISTANCE AND/OR AGRONOMIC TRAITS IN STRAWBERRY PLANTS AND USES THEREOF

(71) Applicant: Plant Sciences Inc., Watsonville, CA (US)

(72) Inventors: Eric JACKSON, Watsonville, CA (US); Michael D. NELSON, Watsonville, CA (US)

(73) Assignee: Plant Sciences Inc., Watsonville, CA (US)

(21) Appl. No.: 17/725,759

(22) Filed: Apr. 21, 2022

Related U.S. Application Data
(60) Provisional application No. 63/177,640, filed on Apr. 21, 2021.
(60) Provisional application No. 63/177,664, filed on Apr. 21, 2021.
(60) Provisional application No. 63/177,676, filed on Apr. 21, 2021.

(51) Int. Cl. C12Q 1/6895; A01H 1/04; A01H 5/08

(52) U.S. Cl. CPC C12Q 1/6895; A01H 1/04; A01H 5/08; C12Q 2600/13

(57) ABSTRACT

The invention relates to single nucleotide polymorphism (SNP) markers that provide improved resistance to one or more diseases and/or improved agronomic traits, such as improved resistance to Fusarium infection, improved resistance to Macrophomina infection, improved resistance to Colletotrichum resistance, improved remontancy and/or improved berry weight. Also described are compositions and methods of using the SNP markers in selecting, obtaining, or developing strawberry plants or varieties with the given improved traits.

### CROSS REFERENCES TO RELATED APPLICATIONS

This application claims the benefit of U.S. Provisional Patent Application No. 63/177,640, filed on Apr. 21, 2021, U.S. Provisional Patent Application No. 63/177,664, filed on Apr. 21, 2021, and U.S. Provisional Patent Application No. 63/177,676, filed on Apr. 21, 2021, which are herein incorporated by reference in their entirety.

### STATEMENT REGARDING ELECTRONIC SUBMISSION OF A SEQUENCE LISTING

A Sequence Listing in ASCII text format, submitted under 37 C.F.R. § 1.821, entitled Sequence_Listing, 25.7 KB in size, generated on Apr. 20, 2022 and filed via EFS-Web. This Sequence Listing is hereby incorporated by reference into the specification for its disclosures.

### FIELD OF THE INVENTION

The present invention is directed generally to molecular markers for identification of certain disease resistance and/or agronomic traits in strawberry plants and uses thereof.

### BACKGROUND OF THE INVENTION

Strawberry plants (Fragaria fragaria x ananassa) are grown throughout the world, both commercially and by individuals, for production of strawberry fruit. Breeders of strawberries often seek to develop strawberry plant varieties with improved traits, such as disease resistance traits or agronomic traits.

One disease that is important to the strawberry industry is Fusarium Wilt, which is caused by one or more species of the Fusarium fungus, including Fusarium oxysporum f. sp. fragariae. As such, it is desired within the strawberry industry to identify and/or develop varieties of strawberry plants that have natural resistance to these fungal species, and therefore natural resistance to Fusarium Wilt disease. The present application provides molecular markers useful for identifying strawberry plants with improved Fusarium resistance and methods of using such markers to identify and/or develop resistant varieties.

Another fungal pathogen that is important to the strawberry industry is Macrophomina phaseolina, which causes Macrophomina Crown Rot or Charcoal Rot in infected plants. As such, it is desired within the strawberry industry to identify and/or develop varieties of strawberry plants that have natural resistance to this fungal species, and therefore natural resistance to Macrophomina Crown Rot and Charcoal Rot disease.

Yet another fungal disease that is important to the strawberry industry is Anthracnose Crown Rot, which is caused by several related species of the fugus Colletotrichum, including Collectotrichum acutatum. As such, it is desired within the strawberry industry to identify and/or develop varieties of strawberry plants that have natural resistance to these fungal species, and therefore natural resistance to the diseases Anthracnose Crown Rot of strawberry.

The present application provides molecular markers useful for identifying strawberry plants with improved Macrophomina resistance and/or Collectotrichum resistance and methods of using such markers to identify and/or develop resistant varieties.

Two agronomic traits that are important to the strawberry industry are remontancy and berry weight. Remontancy refers to a plant's ability to flower more than once during the course of a growing season, which is often referred to as repeat flowering. In strawberry plants, repeated flowering throughout the growing season results in continual berry production throughout the growing season, which is often a desired trait. With regard to berry weight, increased berry weight is often a desired trait as it results in increased overall yield. The present application provides molecular markers useful for identifying strawberry plants with remontancy and/or increased berry weight and methods of using such markers to identify and/or develop varieties with these agronomic traits.

Further aspects, features and advantages of the present invention will be better appreciated upon a reading of the following detailed description of the invention and claims.

### SUMMARY OF INVENTION

Compositions and methods for identifying, selecting, and producing strawberry plants having molecular markers for identification of certain disease resistance, such as resistance to diseases caused by Fusarium, Macrophomia, and/or Colletotrichum, and/or for identification of agronomic traits, such as remontancy and/or increased berry weight, are provided. Strawberry plants and/or strawberry germplasm and/or parts thereof having molecular markers for identification of Fusarium resistance, Macrophomia resistance, Colletotrichum resistance, remontancy, and/or increased berry weight are also provided.

One or more embodiments relate to at least one, at least two, at least three, at least four, at least five, at least six, or at least seven Fusarium resistance markers for a strawberry plant, wherein the Fusarium resistance marker(s) are one or more SNPs as shown in Table 1. The SNPs shown in Table 1 represent the alleles conferring improved resistance to disease caused by Fusarium infection (“Fusarium resistance allele”). Other embodiments relate to one or two Macrophomia resistance markers for a strawberry plant, wherein the Macrophomia resistance marker(s) are one or two of the SNPs as shown in Table 2. The SNPs shown in Table 2 represent the alleles conferring improved resistance to disease caused by Macrophoia infection (“Macrophomina resistance allele”). Other embodiments relate to the Colletotrichum resistance marker for a strawberry plant, wherein the Colletotrichum resistance marker is shown in Table 3. The SNP shown in Table 3 represents the allele conferring improved resistance to disease caused by Colleotrichum infection (“Colletotrichum resistance allele”). Other embodiments relate to a heterozygous state of the remontancy marker for a strawberry plant, wherein the remontancy marker is shown in Table 4. The SNP shown in Table 4 when in heterozygous state (the strawberry plant has a copy of the SNP with a G at the polymorphism loci and a copy of the SNP with a T at the polymorphism loci) represents the allele conferring improved remontancy (“remontancy allele”). Other embodiments relate to at least one, at least two, at least three, at least four, at least five, or at least six berry weight markers for a strawberry plant, wherein the berry weight marker(s) are one or more SNPs as shown in Table 5. The SNPs shown in Table 5 represent the alleles conferring improved berry weight (“berry weight allele”).

One or more embodiments relate to a strawberry plant seed, a strawberry plant, a strawberry plant cultivar, a method for producing a strawberry plant or strawberry plant cultivar, and a method for selecting a strawberry plant or strawberry plant cultivar having at least one, at least two, at least three, at least four, at least five, at least six, or at least seven Fusarium resistance markers for a strawberry plant, wherein the Fusarium resistance marker(s) are one or more SNPs as shown in Table 1; having one or two Macrophomia resistance markers for a strawberry plant, wherein the Macrophomia resistance marker(s) are one or two of the SNPs as shown in Table 2; having the Colletotrichum resistance marker for a strawberry plant, wherein the Colletotrichum resistance marker is shown in Table 3; having the remontancy marker for strawberry plant, wherein the remontancy marker is shown in Table 4; and/or having at least one, at least two, at least three, at least four, at least five, or at least six berry weight markers for a strawberry plant, wherein the berry weight marker(s) are one or more SNPs as shown in Table 5.

One or more embodiments relate to methods for producing or selecting a strawberry plant with improved resistance to Fusarium infection, improved resistance to Macrophomia infection, improved resistance to Colletotrichum infection, improved remontancy, and/or improved berry weight comprising providing an initial population of strawberry plants, obtaining a nucleic acid sample from one or more individuals within said initial population; detecting in each of said nucleic acid samples the presence or absence of one or more SNPs as shown in Tables 1-5, and selecting a strawberry plant from said initial population based on the presence of the one or more SNPs as shown in Table 1-5.

Further embodiments relate to methods for producing or selecting a strawberry plant with improved resistance to Fusarium infection, improved resistance to Macrophomia infection, improved resistance to Colletotrichum infection, improved remontancy, and/or improved berry weight comprising crossing two parental strawberry plants to produce progeny and then detecting in the progeny for the presence or absence of one or more SNPs as shown in Tables 1-5.

Further embodiments relate to methods for producing or selecting a strawberry plant with improved resistance to Fusarium infection, improved resistance to Macrophomia infection, improved resistance to Colletotrichum infection, improved remontancy, and/or improved berry weight comprising crossing a selected strawberry plant that has one or more SNPs as shown in Tables 1-5 with a second strawberry plant to produce progeny plants with improved resistance to Fusarium infection, improved resistance to Macrophomia infection, improved resistance to Colletotrichum infection, improved remontancy, and/or improved berry weight.

Other embodiments relate to methods for producing or selecting a strawberry plant that is homozygous for one or more SNPs as shown in Tables 1-3 and 5.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A provides a table of the disease resistance SNPs that were developed.

FIG. 1B and FIG. 1C provides tables of the remontancy and berry weight SNPS that were developed.

FIG. 2A-FIG. 2D provides the nucleotide sequence of the genome loci containing the identified SNP markers.

FIG. 3 provides a comparison of selectivity using the Fusarium SNP markers identified herein, comparing 2 markers versus 8 markers.

FIG. 4A-FIG. 4D provides the primer and probe sequences utilized to assay certain identified SNPs.

FIG. 5A-FIG. 5B provides the results of a validation study utilizing certain of the identified SNP markers.

FIG. 6 provides the results of a validation study utilizing the identified SNP markers for berry weight.

### DETAILED DESCRIPTION OF THE INVENTION

### Definitions

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated item or group of items but not the exclusion of any other item or group of items and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “molecular marker” or “marker” as used herein refers generally to a molecule, including a gene, protein, carbohydrate structure, or glycolipid, the expression of which in or on a mammalian tissue or cell or secreted can be detected by known methods (or methods disclosed herein) and is predictive or can be used to predict (or aid prediction) the presence or absence of a trait, such as improved resistance to certain pathogens or diseases or agronomic characteristics.

A “single nucleotide polymorphism”, or “SNP”, refers to a single base position in an RNA or DNA molecule (e.g., a polynucleotide), at which different alleles, or alternative nucleotides, exist in a population. The SNP position (interchangeably referred to herein as SNP, SNP site, SNP locus) is usually preceded by and followed by conserved sequences of the allele (e.g., sequences that vary in less than 1/100 or 1/1000 members of the populations). An individual can be homozygous or heterozygous for an allele at each SNP position.

### Molecular Markers for Identification of Certain Disease Resistance and/or Agronomic Traits in Strawberry Plants

### Fusarium Resistance Markers for Strawberry Plants

Fusarium resistance markers for strawberry plants are identified as one or more of the following SNPs shown in Table 1. The underlined base in the corresponding sequence indicates the polymorphism of the SNP.

TABLE 1

| SNP Locus | Sequence |
|---|---|
| PSI-D1 | TGATTGTCTGAGAA (SEQ ID NO: 1) |
| PSI-D2 | TGAAGTATGAATTAA (SEQ ID NO: 2) |
| PSI-D3 | AAGGGCCATGTG (SEQ ID NO: 3) |
| PSI-D4 | CACCGCCTGGTT (SEQ ID NO: 4) |
| PSI-D5 | AACCATCCACAA (SEQ ID NO: 5) |
| PSI-D6 | TGGCCTCCGAG (SEQ ID NO: 6) |
| PSI-D7 | CAGCTTTGGAAT (SEQ ID NO: 7) |

### Macrophomina Resistance Markers for Strawberry Plants

Macrophomina resistance markers for strawberry plants are identified as one or more of the following SNPs shown in Table 2. The underlined base in the corresponding sequence indicates the polymorphism of the SNP.

TABLE 2

| SNP Locus | Sequence |
|---|---|
| PSI-D11 | ATGCATAAGATGTG (SEQ ID NO: 8) |
| PSI-D12 | ACTGTAACGACA (SEQ ID NO: 9) |

### Colletotrichum Resistance Marker for Strawberry Plants

Colletotrichum resistance marker for strawberry plants is identified as the following SNP shown in Table 3. The underlined base in the corresponding sequence indicates the polymorphism of the SNP.

TABLE 3

| SNP Locus | Sequence |
|---|---|
| PSI-D8 | AGATAGCTAAACTCA (SEQ ID NO: 10) |

### Remontancy Marker for Strawberry Plants

The remontancy marker for strawberry plants is identified as the following SNP shown in Table 4, wherein the remontancy allele is a heterozygous state of the following SNP. The bracketed bases in the corresponding sequence indicate the polymorphism of the SNP.

TABLE 4

| SNP Locus | Sequence |
|---|---|
| PSI-ROM 1 | GGATC[G/T]GTGAAG (SEQ ID NO: 11) |

### Improved Berry Weight Marker for Strawberry Plants

Improved berry weight markers for strawberry plants are identified as one or more of the following SNPs shown in Table 5. The underlined base in the corresponding sequence indicates the polymorphism of the SNP.

TABLE 5

| SNP Locus | Sequence |
|---|---|
| PSI-BW1 | CTCGCCGTTCTA (SEQ ID NO: 12) |
| PSI-BW2 | GATCYACATTCT (SEQ ID NO: 13) |
| PSI-BW3 | ATCCTTATCRAC (SEQ ID NO: 14) |
| PSI-BW4 | TGGTGGTTGTTC (SEQ ID NO: 15) |
| PSI-BW5 | GCCACGAAATGA (SEQ ID NO: 16) |
| PSI-BW6 | AAGCATCATCAA (SEQ ID NO: 17) |

### Exemplified Embodiments

Embodiment 1. A method for selecting a strawberry plant with improved resistance to Fusarium infection, the method comprising:
a. providing an initial population of strawberry plants;
b. obtaining a nucleic acid sample from one or more individuals within said initial population;
c. detecting in each of said nucleic acid samples the presence or absence of two or more alleles conferring improved resistance to disease caused by Fusarium infection (“Fusarium resistance allele”), wherein each of the two or more Fusarium resistance alleles is an allele of at least one single nucleotide polymorphism (SNP) marker selected from the SNP loci within SEQ ID NOs:1-7; and
d. selecting a strawberry plant from said initial population based on the presence of the two or more Fusarium resistance alleles in the nucleic acid sample.

Embodiment 2. A method for selecting a strawberry plant with improved resistance to Fusarium infection, the method comprising:
a. crossing two parental strawberry plants to produce a plurality of progeny seed;
b. growing said progeny seed to produce a population of progeny plants;
c. obtaining a nucleic acid sample from one or more individuals within said population of progeny plants;
d. detecting in each of said nucleic acid samples the presence or absence of two or more alleles conferring improved resistance to disease caused by Fusarium infection (“Fusarium resistance allele”), wherein each of the two or more Fusarium resistance alleles is an allele of at least one single nucleotide polymorphism (SNP) marker selected from the SNP loci within SEQ ID NOs:1-7; and
e. selecting a strawberry plant from said population of progeny plants based on the presence of the two or more Fusarium resistance alleles in the nucleic acid sample.

Embodiment 3. A method for obtaining a strawberry plant with improved resistance to Fusarium infection, the method comprising:
a. providing an initial population of strawberry plants;
b. obtaining a nucleic acid sample from one or more individuals within said initial population;
c. detecting in each of said nucleic acid samples the presence or absence of two or more alleles conferring improved resistance to disease caused by Fusarium infection (“Fusarium resistance allele”), wherein each of the two or more Fusarium resistance alleles is an allele of at least one single nucleotide polymorphism (SNP) loci within SEQ ID NOs:1-7;
d. selecting a strawberry plant from said initial population based on the presence of the two or more Fusarium resistance alleles in the nucleic acid sample; and
e. crossing the selected strawberry plant with a second strawberry plant to produce progeny plants comprising improved resistance to disease caused by Fusarium infection.

Embodiment 4. The method of embodiment 3, wherein two strawberry plants are selected from said initial population based on the presence of the two or more Fusarium resistance alleles in the nucleic acid sample for each of the two selected plants, and wherein the two selected plants are crossed with one another to produce progeny plants comprising improved resistance to disease caused by Fusarium infection.

Embodiment 5. The method of any one of embodiments 3 or 4, wherein at least one of the two strawberry plants in the cross is homozygous for at least one of the two or more Fusarium resistance alleles.

Embodiment 6. The method of any one of embodiments 3 or 4, wherein both of the two strawberry plants in the cross are homozygous for at least one of the two or more Fusarium resistance alleles.

Embodiment 7. The method of any one of embodiments 1-6, wherein the detection step comprises detecting at least three of the SNP loci within SEQ ID NOs:1-7, optionally at least four of the SNP loci within SEQ ID NOs:1-7, optionally at least five of the SNP loci within SEQ ID NOs:1-7, optionally at least six of the listed SNP loci within SEQ ID NOs:1-7, or optionally at least seven of the SNP loci within SEQ ID NOs:1-7.

Embodiment 8. The method of any one of embodiments 1-7, wherein the detection step further comprises detecting
i) at least one allele conferring improved resistance to disease caused by Macrophomina infection (“Macrophomina resistance allele”), wherein each of the at least one Macrophomina resistance alleles is an allele of at least one single nucleotide polymorphism (SNP) loci within SEQ ID NOs:8 and 9;
ii) an allele conferring improved resistance to disease caused by Colletotrichum infection (“Colletotrichum resistance allele”), wherein the Colletotrichum resistance allele is an allele of the SNP locus within SEQ ID NO:10;
iii) at least one Macrophomina resistance allele of i) and the Colletotrichum resistance allele of ii); or
iv) both Macrophomina resistance alleles of i) and the Colletotrichum resistance allele of ii).

Embodiment 9. The method of any one of embodiments 1-8, wherein the detection step further comprises detecting the presence or absence of an allele conferring remontancy (“remontancy allele”), wherein the remontancy allele is a heterozygous state at a single nucleotide polymorphism (SNP) locus within SEQ ID NO:11.

Embodiment 10. A method for selecting a strawberry plant with improved resistance to Macrophomina and/or Colletotrichum infection, the method comprising:
a. providing an initial population of strawberry plants;
b. obtaining a nucleic acid sample from one or more individuals within said initial population;
c. detecting in each of said nucleic acid samples the presence or absense of two or more alleles conferring improved resistance to disease caused by Macrophomina and/or Colletotrichum infection (“Macrophomina/Colletotrichum resistance allele”), wherein each of the two or more Macrophomina/Colletotrichum resistance alleles is an allele of at least one single nucleotide polymorphism (SNP) marker selected from the SNP loci within SEQ ID NOs:8-10; and
d. selecting a strawberry plant from said initial population based on the presence of the two or more Macrophomina/Colletotrichum resistance allelez in the nucleic acid sample.

Embodiment 11. A method for selecting a strawberry plant with improved resistance to Macrophomina and/or Colletotrichum infection, the method comprising:
a. crossing two parental strawberry plants to produce a plurality of progeny seed;
b. growing said progeny seed to produce a population of progeny plants;
c. obtaining a nucleic acid sample from one or more individuals within said population of progeny plants;
d. detecting in each of said nucleic acid samples the presence or absence of two or more alleles conferring improved resistance to disease caused by Macrophomina and/or Colletotrichum infection (“Macrophomina/Colletotrichum resistance allele”), wherein each of the two or more Macrophomina/Colletotrichum resistance alleles is an allele of at least one single nucleotide polymorphism (SNP) marker selected from the SNP loci within SEQ ID NOs:8-10; and
e. selecting a strawberry plant from said population of progeny plants based on the presence of the two or more Macrophomina/Colletotrichum resistance alleles in the nucleic acid sample.

Embodiment 12. A method for obtaining a strawberry plant with improved resistance to Macrophomina or Colletotrichum infection, the method comprising:
a. providing an initial population of strawberry plants;
b. obtaining a nucleic acid sample from one or more individuals within said initial population;
c. detecting in each of said nucleic acid samples the presence or absence of two or more alleles conferring improved resistance to disease caused by Macrophomina and/or Colletotrichum infection (“Macrophomina/Colletotrichum resistance allele”), wherein each of the two or more Macrophomina/Colletotrichum resistance alleles is an allele of at least one single nucleotide polymorphism (SNP) marker selected from the SNP loci within SEQ ID NOs:8-10;
d. selecting a strawberry plant from said initial population based on the presence of the two or more Macrophomina/Colletotrichum resistance alleles in the nucleic acid sample; and
   crossing the selected strawberry plant with a second strawberry plant to produce progeny plants comprising improved resistance to disease caused by Macrophomina or Colletotrichum infection.

Embodiment 13. The method of embodiment 12, wherein two strawberry plants are selected from said initial population based on the presence of the two or more Macrophomina/Colletotrichum resistance alleles in the nucleic acid sample for each of the two selected plants, and wherein the two selected plants are crossed with one another to produce progeny plants comprising improved resistance to disease caused by Macrophomina or Colletotrichum infection.

Embodiment 14. The method of any one of embodiment 12 or embodiment 13, wherein at least one of the two strawberry plants in the cross is homozygous for at least one of the two or more Macrophomina/Colletotrichum resistance alleles.

Embodiment 15. The method of any one of embodiment 12 or embodiment 13, wherein both of the strawberry plants in the cross are homozygous for at least one of the two or more Macrophomina/Colletotrichum resistance alleles.

Embodiment 16. The method of any one of embodiments 10-15, wherein the detection step comprises detecting three of the SNP loci within SEQ ID NOs:8-10.

Embodiment 17. The method of any one of embodiments 10-16, wherein the detection step further comprises detecting at least one allele conferring improved resistance to disease caused by Fusarium infection (“Fusarium resistance allele”), wherein each of the at least one Fusarium resistance alleles is an allele of at least one single nucleotide polymorphism (SNP) loci within SEQ ID NOs:1-7.

Embodiment 18. The method of embodiment 17, wherein the method comprises detecting two or more Fusarium resistance alleles of SEQ ID NOs:1-7, optionally three or more Fusarium resistance alleles of SEQ ID NOs:1-7, optionally four or more Fusarium resistance alleles of SEQ ID NOs:1-7, optionally five or more Fusarium resistance alleles of SEQ ID NOs:1-7, optionally six or more Fusarium resistance alleles of SEQ ID NOs:1-7, optionally seven or more Fusarium resistance alleles of SEQ ID NOs:1-7.

Embodiment 19. The method of any one of embodiment 10-18, wherein the detection step further comprises detecting the presence or absence of an allele conferring remontancy (“remontancy allele”), wherein the remontancy allele is a heterozygous state at a single nucleotide polymorphism (SNP) locus within SEQ ID NO:11.

Embodiment 20. A method for selecting a strawberry plant with improved agronomic traits, the method comprising:
a. providing an initial population of strawberry plants;
b. obtaining a nucleic acid sample from one or more individuals within said initial population;
c. detecting in each of said nucleic acid samples the presence or absence of an allele conferring remontancy (“remontancy allele”), wherein the remontancy allele is a heterozygous state at a single nucleotide polymorphism (SNP) locus within SEQ ID NO:11; and
d. selecting a strawberry plant from said initial population based on the presence of the at remontancy allele in the nucleic acid samples.

Embodiment 21. A method for selecting a strawberry plant with improved agronomic traits, the method comprising:
a. crossing two parental strawberry plants to produce a plurality of progeny seed;
b. growing said progeny seed to produce a population of progeny plants;
c. obtaining a nucleic acid sample from one or more individuals within said population of progeny plants;
d. detecting in each of said nucleic acid samples the presence of an allele conferring remontancy (“remontancy allele”), wherein the remontancy allele is a heterozygous state at a single nucleotide polymorphism (SNP) locus within SEQ ID NO:11; and
e. selecting a strawberry plant from said population of progeny plants based on the presence of the at remontancy allele in the nucleic acid samples.

Embodiment 22. A method for obtaining a strawberry plant with improved agronomic traits, the method comprising:
a. providing an initial population of strawberry plants;
b. obtaining a nucleic acid sample from one or more individuals within said initial population;
c. detecting in each of said nucleic acid samples the presence of an allele conferring remontancy (“remontancy allele”), wherein the remontancy allele is a heterozygous state at a single nucleotide polymorphism (SNP) locus within SEQ ID NO:11;
d. selecting a strawberry plant from said population of progeny plants based on the presence of the at remontancy allele in the nucleic acid samples; and
e. crossing the selected strawberry plant with a second strawberry plant to produce progeny plants comprising improved agronomic traits.

Embodiment 23. The method of Embodiment 22, wherein two strawberry plants are selected from said initial population based on the presence of the selected allele in the nucleic acid sample for each of the two selected plants, and wherein the two selected plants are crossed with one another to produce progeny plants comprising improved agronomic traits.

Embodiment 24. The method of any one of Embodiments 20-23, wherein the detection step further comprises detecting in each of said nucleic acid samples the presence or absence of at least one allele conferring increased berry weight (“berry weight allele”), wherein the at least one berry weight allele is an allele of at least one SNP loci within SEQ ID NOs:12-17.

Embodiment 25. The method of Embodiment 24, wherein the detection step comprises detecting at least two berry weight alleles of SEQ ID NOs:12-17, optionally at least three berry weight alleles of SEQ ID NOs:12-17, optionally at least four berry weight alleles of SEQ ID NOs:12-17, optionally at least five berry weight alleles of SEQ ID NOs:12-17, or optionally at least six berry weight alleles of SEQ ID NOs:12-17.

Embodiment 26. The method of any one of Embodiment 24 or Embodiment 25, wherein at least one of the two strawberry plants in the cross is homozygous for the selected berry weight alleles.

Embodiment 27. The method of any one of Embodiment 24 or Embodiment 25, wherein both of the strawberry plants in the cross are homozygous for the selected berry weight alleles.

### EXAMPLES

### Background of SNP Analysis and CIPHER™

By combining genotypic and phenotypic analysis, a SNP marker can be correlated with a particular phenotypic trait, such as a disease resistance trait or agronomic trait. By identifying multiple SNP markers that associate with the same phenotypic trait, it is possible to combine multiple SNP markers into a haplotype that associates even more closely with the desired phenotypic trait.

CIPHER™ is a technology that identifies a set of SNP markers that can be used, either alone or in combination, to identify, with a high likelihood of success, a particular phenotypic trait among individuals in a population. CIPHER™ uses sequencing of reduced genomes along with measurements of key traits to generate information rich DNA-based profiles across plant populations using machine learning algorithms. The analysis software then rigorously selects a set of key DNA-based predictors and builds a CIPHER™ algorithm that decrypts the predictors into qualitative haplotype (categorical) values and quantitative (numeric) scores for a specific trait. Diagnostic molecular assays using novel DNA extraction with conventional and isothermal polymerase chain reaction (PCR) are designed by the analysis process and the CIPHER™ is physically validated under a blind test in the laboratory setting.

### Example 1: Developing and Implementing Fusarium Resistance SNPs and CIPHER™

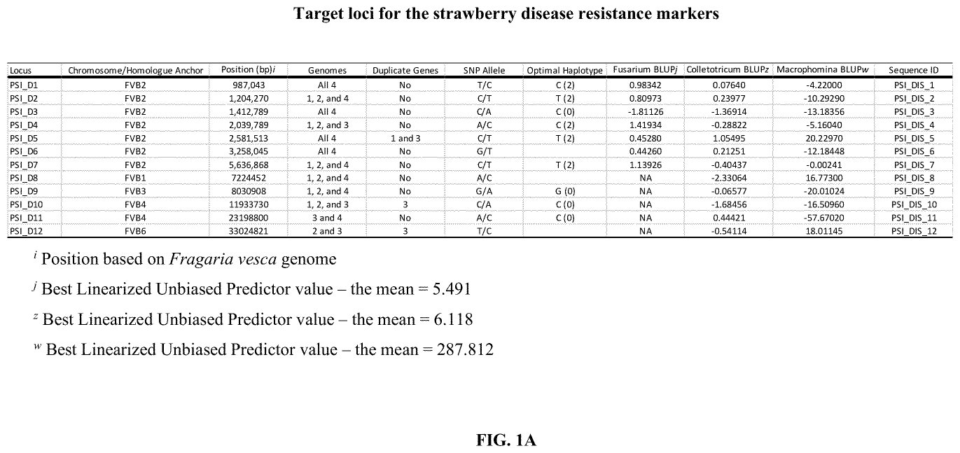

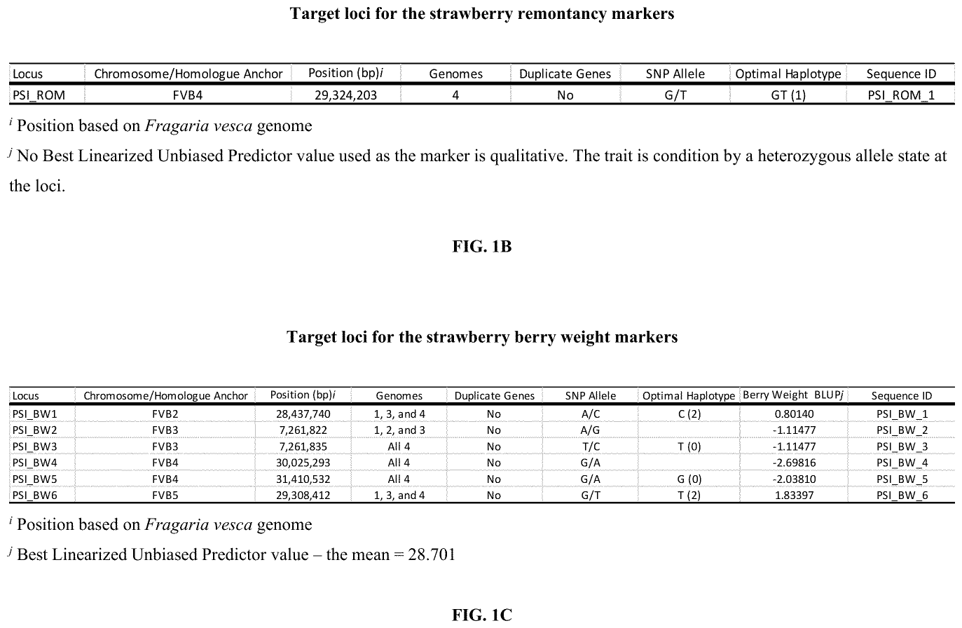

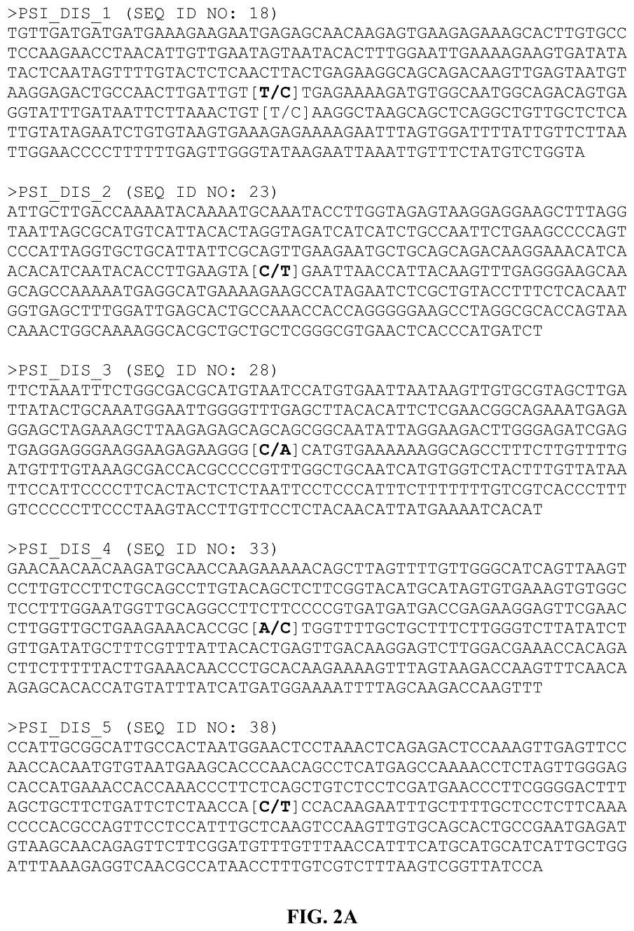

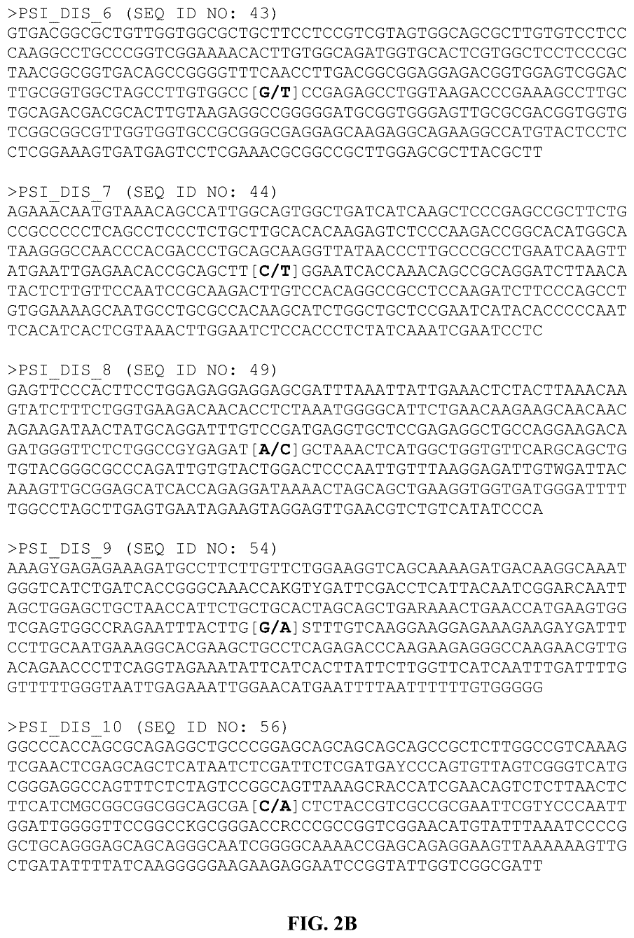

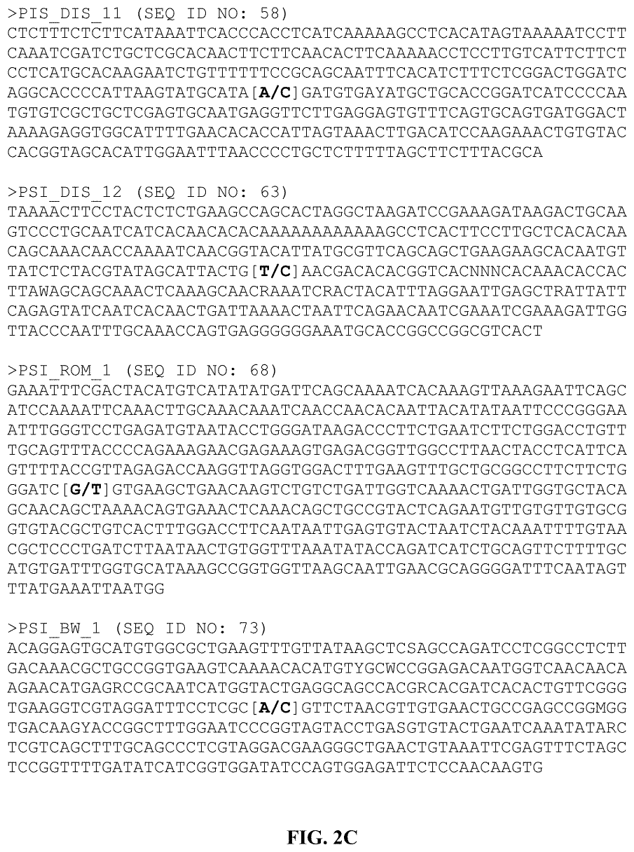

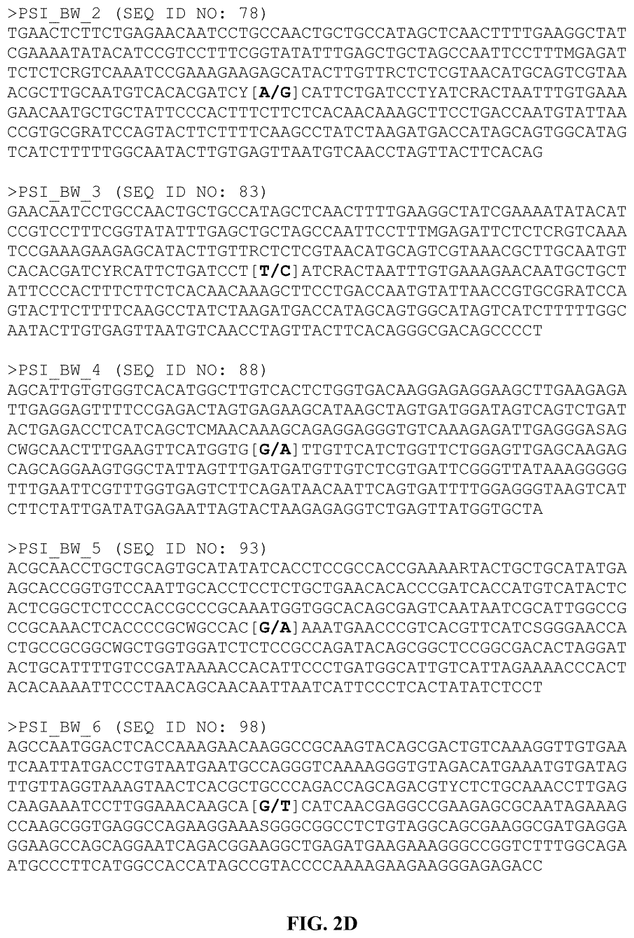

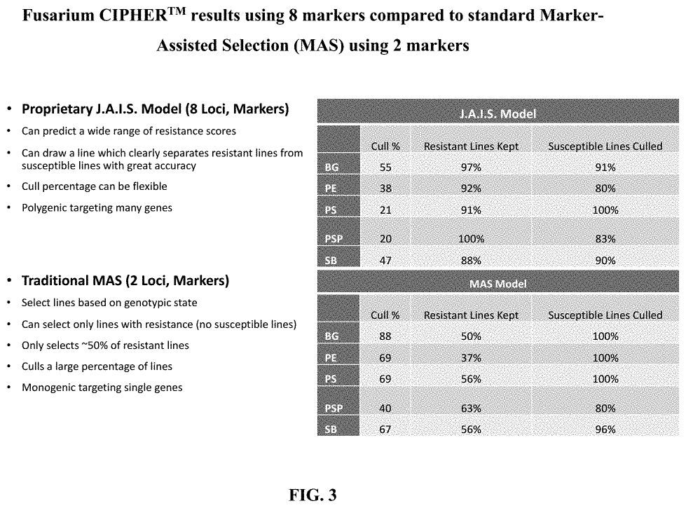

SNPs were identified and CIPHERs™ were developed that are capable of identifying, with high accuracy, strawberry plants that possess improved resistance to Fusarium disease, for example disease caused by Fusarium oxysporum f. sp. fragariae, and, conversely, susceptible plants. The CIPHERs™ are based on decryption algorithms which allow qualitative and quantitative scores to be generated from seven loci or predictors for Fusarium (FIGS. 1, 2A, and 2B). By including an increasing number of SNPs in the selection criteria, and depending on which combined set of SNPs is selected, it is possible to have a much more selective determination of resistant versus susceptible lines than can be achieved by using only one or a few SNPs. Fusarium resistance haplotypes and scores were predicted for over 1,800 strawberry lines from five different breeding programs. Comparing the predicted scores to 300 known measured Fusarium resistance scores in a blind test, a threshold could be discerned in each of the 5 programs which separated resistant lines from susceptible lines with over 88% accuracy on average. A traditional marker-assisted-selection method based on two markers that can accurately identify resistant lines based on genotypic state (present/absent) was used to select resistant lines. This method was only able to select just over 52% of the total known resistant lines on average (FIG. 3).

The disease CIPHERs™ were developed by carefully selecting a training set of lines. The training set was evaluated for resistance to Fusarium sp., M phaseolina, and C. acutatum under controlled greenhouse conditions over three years (2017, 2018, 2020). The mean disease severity (phenotypes) for each line in the training set were combined with the corresponding cipheralleles (genotypes) and the software developed a preliminary prediction algorithm using its proprietary adaptive learning network. The prediction algorithm was then validated using both cross validation with the appropriate hold-out strategy for the trait type and physical validation. The algorithm was then optimized using a proprietary selection network to identify the smallest subset of predictors or alleles that accurately predict the trait.

The Fusarium CIPHER™ Algorithm:

y̅=μ+i^{1*α}+(i^{2*ρ})²+i^{3*γ}+i^{4*δ}+i^{5*ε}+i^{6θ}+i^{7*β}

where predicted trait or attribute value (y̅) is a derivative of the dynamic mean (μ) plus the numeric state of the CIPHER™ allele (ith) times the corresponding J.A.I.S. linearized unbiased prediction value for the ith allele (α, β, γ, δ, ε, θ, ρ) was used to impute values for over 1,800 lines in the programs based on the following calculus:

y̅=5.491+i^{1*[0.983]}(i^{2*[0.810]})²+i^{3*[-1.813]}+i^{4*[1.149]}+i^{5*[0.453]}+i^{6[0.443]}+i^{7*[1.139]}

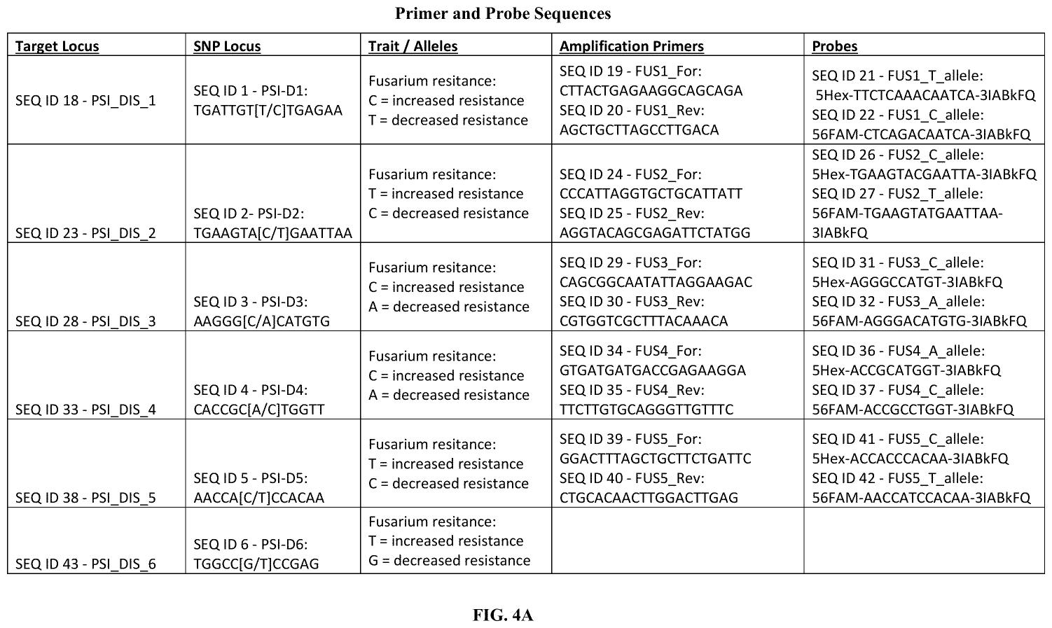

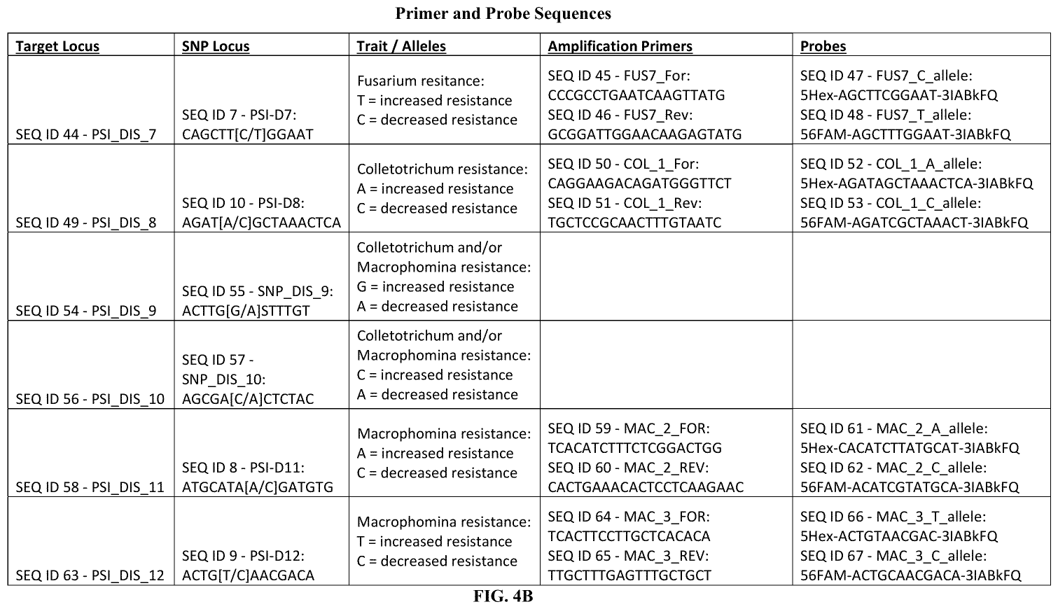

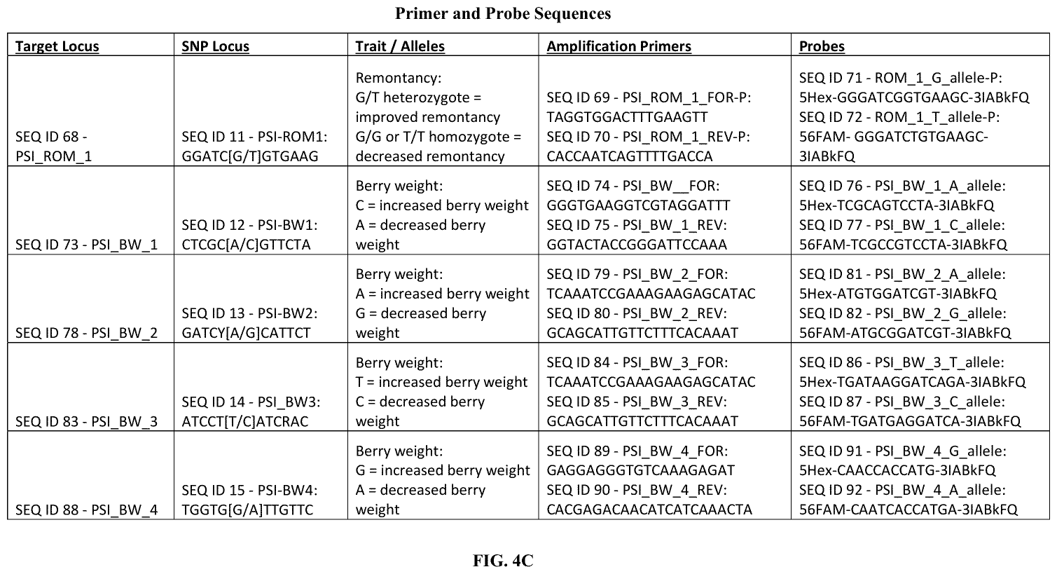

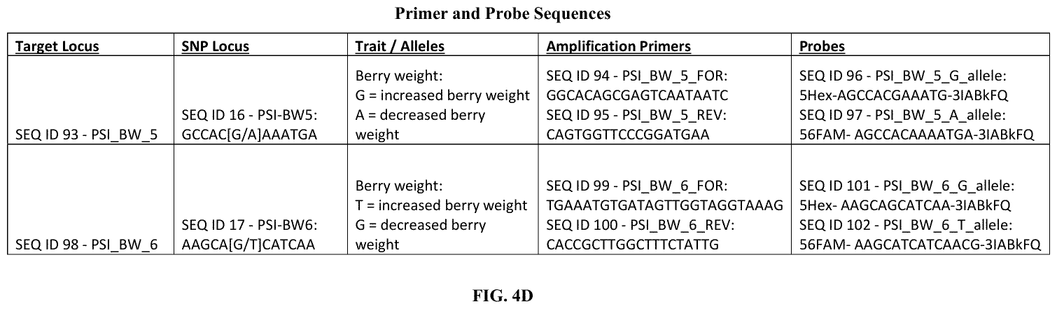

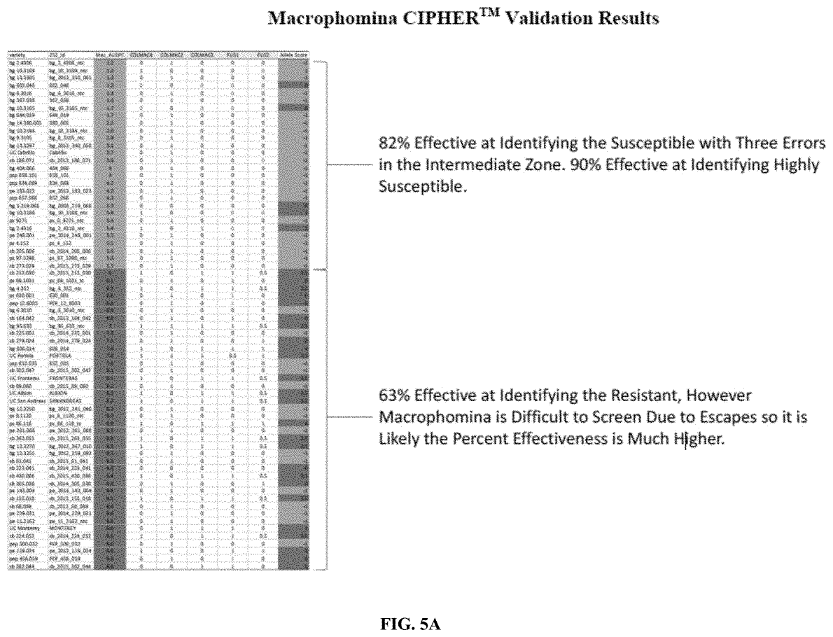

The sequence information from each locus (FIG. 2A and FIG. 2B) was used to create the Trait CIPHER™ Assays using molecular beacon technology (FIGS. 4A-4D). In brief, forward and reverse primers were designed to amplify the target region containing the single nucleotide polymorphism (SNP). Two different forward primers were designed, one specific to each of the SNP alleles in question. The forward primers are differentiated by a short sequence on the 5′ end that does not match the template DNA but is homologous to a secondary probe sequence that contains a specific fluorophore molecule. Once the sequence is amplified during Polymerase Chain Reaction (PCR), the probe corresponding to the SNP allele in the sample is incorporated into the growing template and the fluorophore is released. The free fluorophore is then read using a spectrophotometer and the SNP allele is differentiated.

The imputed trait or attribute values of a test set (N=300 lines) representing five breeding programs that were not screened in the greenhouse experiments were compared to actual disease severity measurements using the Trait CIPHER™ Assays. The comparisons yielded an accuracy of 88% or greater, showing greater than 97% accuracy for Fusarium. When comparing this result to the traditional marker-assisted-selection method currently being deployed, the Fusarium CIPHER™ was on average 40.8% more accurate.

The Fusarium CIPHER™ was used to cull out Fusarium susceptible lines across the breeding programs (N˜150,000 seedlings) prior to transplanting from the greenhouse to the field. In brief, F₁ seeds were planted in trays and incubated in the greenhouse. Three-millimeter leaf disks were cut from each seedling and placed in a 96-well plate maintaining the line identification using a plate record key. The plates were sent in for Trait CIPHER™ sequencing and the data was uploaded to a database. A workflow utilizing the Fusarium CIPHER™ Algorithm deciphered the data for each line into predicted Fusarium haplotype and severity scores. A culling tool was utilized to set the minimum Fusarium severity score desired and view the overall metrics for percent cull, maximum/minimum severity, and unique severity scores for each cross. Once the desired culling percent was determined via evaluation of the metrices, the wells in each tray containing a line to be culled was identified using a proprietary algorithm embedded in the workflow. In addition, individual tray maps were constructed to guide physically removing the unwanted lines from the trays while consolidating the good lines.

The overall accuracy of the CIPHER™ algorithm was re-evaluated based on the assumed segregation ratios for each cross and by testing selected lines in the greenhouse under artificial Fusarium disease pressure. Overall, the expected segregation ratios were calculated for the crosses and the selected lines fit the expected disease response greater than 98% of the time.

A close evaluation of the haplotype representation of all seven allele states that make up the Fusarium CIPHER™ revealed that the optimal allele state:

y̅=5.491+2*0.983+(2*0.810)²+0*−1.812+2*419+2*0.453+2*0.443+2*1.139

does not exist in the breeding programs. The theoretical resistance of the optimal haplotype would be 1.5 times greater than currently possible. The program fixes the alleles utilizing the Trait CIPHER™. The Fusarium CIPHER™ can be used to screen a global strawberry population to confirm the uniqueness of this optimal haplotype.

### Example 2: Developing and Implementing Macrophomina/Colletotrichum Resistance SNPs and CIPHER™

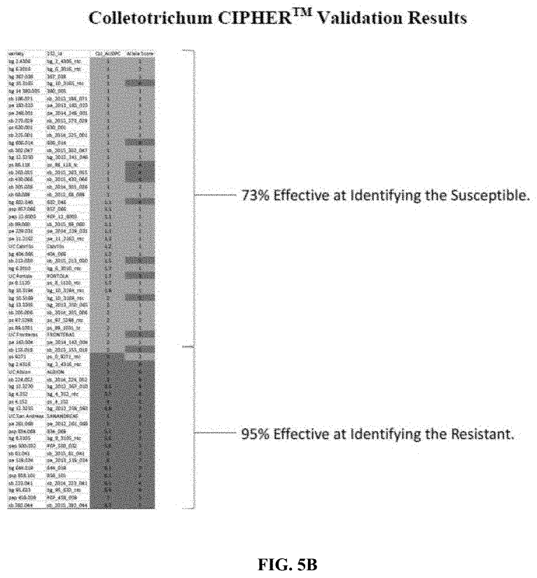

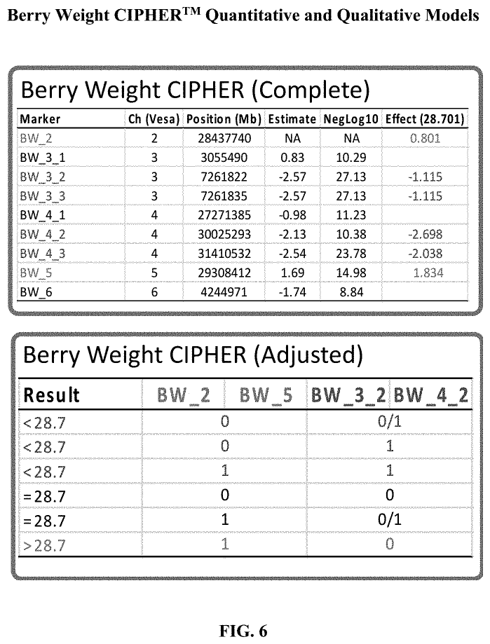

SNPs were identified and CIPHERs™ were developed that are capable of identifying, with high accuracy, strawberry plants that possess improved resistance to Macrophomina and/or Colletotrichum disease, for example disease caused by Macrophomina phaseolina or Colletotrichum acutatum, and, conversely, susceptible plants. The CIPHERs™ are based on decryption algorithms which allow qualitative and quantitative scores to be generated from five loci or predictors for Macrophomina and Colletotrichum (FIGS. 1, 2B, and 2C). By including an increasing number of SNPs in the selection criteria, and depending on which combined set of SNPs is selected, it is possible to have a much more selective determination of resistant versus susceptible lines than can be achieved by using only one or a few SNPs. Macrophomina and Colletotrichum resistance haplotypes and scores were predicted for over 1,800 strawberry lines from five different breeding programs. Haplotypes and allele scores generated from the prediction algorithm were compared across 70 lines for both Macrophomina and Colletotrichum in a 10 blind test. The results indicated the Macrophomina CIPHER™ could identify susceptible lines with and accuracy of 82% and resistant lines with and accuracy of 63% (FIG. 5A), while the Colletotrichum CIPHER™ could identify susceptible lines with and accuracy 73% of and resistant lines with and accuracy of 95% (FIG. 5B). The disease CIPHERs™ were developed by carefully selecting a training set of lines. The training set was evaluated for resistance to Fusarium sp., M. phaseolina, and C. acutatum under controlled greenhouse conditions over three years (2017, 2018, 2020). The mean disease severity (phenotypes) for each line in the training set were combined with the corresponding cipheralleles (genotypes) and the software developed a preliminary prediction algorithm using its proprietary adaptive learning network. The prediction algorithm was then validated using both cross validation with the appropriate hold-out strategy for the trait type and physical validation. The algorithm was then optimized using a proprietary selection network to identify the smallest subset of predictors or alleles (FIG. 2) that accurately predict the trait.

### Macrophomina:

$$y = 287.812 + {j}^{1 ⋆ - 4.22} + {i}^{2 ⋆ - 10.29} + {i}^{3 ⋆ - 13.18} + {i}^{4 ⋆ - 5.16} + {i}^{5 ⋆ 20.23} + {i}^{6 ⋆ - 12.18} + {i}^{7 ⋆ - 0.002} + {i}^{8 ⋆ 16.77} + {i}^{9 ⋆ - 20.01} + {i}^{10 ⋆ - 16.51} + {i}^{11 ⋆ - 57.67} + {i}^{12 ⋆ 10.01}$$

### Colletotrichum:

$$y = 6.118 + {j}^{1 ⋆ 0.08} + {i}^{2 ⋆ 0.24} + {i}^{3 ⋆ - 1.37} + {i}^{4 ⋆ - 0.29} + {i}^{5 ⋆ 1.05} + {i}^{6 ⋆ 0.21} + {i}^{7 ⋆ - 0.4} + {i}^{8 ⋆ - \text{?}} + {i}^{9 ⋆ - 0.66} + {i}^{10 ⋆ - 1.68} + {i}^{11 ⋆ 0.44} + {i}^{12 ⋆ - 0.54}$$

$$\text{?} \text{indicates text missing or illegible when filed}$$

The sequence information from each locus (FIG. 2) was used to create the Trait CIPHER™ Assays using molecular beacon technology (FIGS. 4A-4D). In brief, forward and reverse primes were designed to amplify the target region containing the single nucleotide polymorphism (SNP). Two different forward primers were designed, one specific to each of the SNP alleles in question. The forward primers are differentiated by a short sequence on the 5′ end that does not match the template DNA but is homologous to a secondary probe sequence that contains a specific fluorophore molecule. Once the sequence is amplified during Polymerase Chain Reaction (PCR), the probe corresponding to the SNP allele in the sample is incorporated into the growing template and the fluorophore is released. The free fluorophore is then read using a spectrophotometer and the SNP allele is differentiated.

The imputed trait or attribute values of a test set (N=300 lines) representing five breeding programs that were not screened in the greenhouse experiments were compared to actual disease severity measurements using the Trait CIPHER™ Assays. The optimal allele states were calculated for:

### Macrophomina:

$$y = 28 \; 7.8 \; 12 + {j}^{1 ( 0 ⋆ - 4.22 )} + {i}^{2 ( 0 ⋆ - 10.29 )} + {i}^{3 ( 0 ⋆ - 13.18 )} + {i}^{4 ( 0 ⋆ - 5.16 )} + {i}^{5 ( 2 ⋆ 20.23 )} + i {6}^{( 0 ⋆ - 12.18 )} + i {7}^{( 0 ⋆ - 0.002 )} + i {8}^{( 2 ⋆ 16.77 )} + i {9}^{( 0 ⋆ - 20.01 )} + i {10}^{( 0 ⋆ - 116.51 )} + i {11}^{( 0 ⋆ 57.67 )} + {i}^{12 ( 2 \cdot 18.01 )}$$

### Colletotrichum:

$$y = 6.118 + {j}^{1 ( 2 ⋆ 0.08 )} + {i}^{2 ( 2 ⋆ 0.24 )} + {i}^{3 ( 0 ⋆ - 1.37 )} + {i}^{4 ( 0 ⋆ - 0.29 )} + {i}^{5 ( 2 ⋆ 1.05 )} + i {6}^{( 2 ⋆ 0.21 )} + i {7}^{( 0 ⋆ - 0.4 )} + i {8}^{( 0 ⋆ - 2.33 )} + i {9}^{( 0 ⋆ - 0.66 )} + i {10}^{( 0 ⋆ - 1.68 )} + i {11}^{( 2 ⋆ 0.44 )} + {i}^{12 ( 0 \cdot - 0.54 )}$$

And were not found within the programs.

### Example 3: Developing and Implementing Agronomic Trait SNPs and CIPHER™

SNPs were identified and CIPHERs™ were developed that are capable of identifying, with high accuracy, strawberry plants that express remontancy and increased berry weight, and, conversely, those plants that do not. By including an increasing number of SNPs in the selection criteria, and depending on which combined set of SNPs is selected, it is possible to have a much more selective determination of lines possessing the desired agronomic traits than can be achieved by using only one or a few SNPs. A single locus was discovered where in the heterozygous state conditioned remontancy (FIGS. 1, 2C and 2D). The CIPHER™ was tested across over 500 lines under blind test and was shown to have an accuracy of over 90%.

The Berry weight CIPHER™ included six loci that associate with this phenotypic trait (FIGS. 1 and 2):

y̅=μ+j^{1*α}+i^{2*ρ}+^{3*γ}+i^{4*δ}+i^{5*ε}+i^{6*θ}

where predicted trait or attribute value (y̅) is a derivative of the dynamic J.A.I.S. mean (μ) plus the numeric state of the CIPHER™ allele (ith) times the corresponding J.A.I.S. linearized unbiased prediction value for the ith allele (α, β, γ, δ, ε, θ) was used to impute values for over 1,800 lines in the PSI programs based on the following calculus:

y̅=28.71+j^{12*0.80}+i^{2*-1.11}+i^{3*-1.11}+i^{4*-2.70}+i^{5*-2.04}+i^{6*1.83}

In addition, the optimal allele state was used which allowed three classes of discrimination including greater than, equal to, or less than 28-gram berries with an accuracy of 85% (FIG. 6). The optimal allele state:

y=28.71+i^{1(0*0.80)}+i^{2(0*-1.11)}+i^{3(0*-1.11)}+i^{4(0*-2.70)}+i^{5(0*-2.04)}+i^{6(2*1.83)}

was not found across the breeding programs.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All documents cited herein are incorporated by reference.

We claim:

1. (canceled)

2. A method for selecting a strawberry plant with improved resistance to Fusarium infection, the method comprising:
a. crossing two parental strawberry plants to produce a plurality of progeny seed;
b. growing said progeny seed to produce a population of progeny plants;
c. obtaining a nucleic acid sample from one or more individuals within said population of progeny plants;
d. detecting in each of said nucleic acid samples the presence or absence of two or more alleles conferring improved resistance to disease caused by Fusarium infection (“Fusarium resistance allele”), wherein each of the two or more Fusarium resistance alleles is an allele of at least one single nucleotide polymorphism (SNP) marker selected from the SNP loci within SEQ ID NOs:1-7; and
e. selecting a strawberry plant from said population of progeny plants based on the presence of the two or more Fusarium resistance alleles in the nucleic acid sample.

3. A method for obtaining a strawberry plant with improved resistance to Fusarium infection, the method comprising:
a. providing an initial population of strawberry plants;
b. obtaining a nucleic acid sample from one or more individuals within said initial population;
c. detecting in each of said nucleic acid samples the presence or absence of two or more alleles conferring improved resistance to disease caused by Fusarium infection (“Fusarium resistance allele”), wherein each of the two or more Fusarium resistance alleles is an allele of at least one single nucleotide polymorphism (SNP) loci within SEQ ID NOs:1-7;
d. selecting a strawberry plant from said initial population based on the presence of the two or more Fusarium resistance alleles in the nucleic acid sample; and
e. crossing the selected strawberry plant with a second strawberry plant to produce progeny plants comprising improved resistance to disease caused by Fusarium infection.

4. The method of claim 3, wherein two strawberry plants are selected from said initial population based on the presence of the two or more Fusarium resistance alleles in the nucleic acid sample for each of the two selected plants, and wherein the two selected plants are crossed with one another to produce progeny plants comprising improved resistance to disease caused by Fusarium infection.

5. The method of claim 3, wherein the selected strawberry plant is homozygous for at least one of the two or more Fusarium resistance alleles.

6. (canceled)

7. The method of claim 3, wherein the detection step comprises detecting three, four, five, or six of the SNP loci within SEQ ID NOs:1-7.

8. The method of claim 3, wherein the detection step further comprises detecting
i) at least one allele conferring improved resistance to disease caused by Macrophomina infection (“Macrophomina resistance allele”), wherein each of the at least one Macrophomina resistance alleles is an allele of at least one single nucleotide polymorphism (SNP) loci within SEQ ID NOs:8 and 9;
ii) an allele conferring improved resistance to disease caused by Colletotrichum infection (“Colletotrichum resistance allele”), wherein the Colletotrichum resistance allele is an allele of the SNP locus within SEQ ID NO:10;
iii) at least one Macrophomina resistance allele of i) and the Colletotrichum resistance allele of ii); or
iv) both Macrophomina resistance alleles of i) and the Colletotrichum resistance allele of ii).

9. The method of claim 3, wherein the detection step further comprises detecting the presence or absence of an allele conferring remontancy (“remontancy allele”), wherein the remontancy allele is a heterozygous state at a single nucleotide polymorphism (SNP) locus within SEQ ID NO:11.

10. (canceled)

11. A method for selecting a strawberry plant with improved resistance to Macrophomina and/or Colletotrichum infection, the method comprising:
a. crossing two parental strawberry plants to produce a plurality of progeny seed;
b. growing said progeny seed to produce a population of progeny plants;
c. obtaining a nucleic acid sample from one or more individuals within said population of progeny plants;
d. detecting in each of said nucleic acid samples the presence or absence of two or more alleles conferring improved resistance to disease caused by Macrophomina and/or Colletotrichum infection (“Macrophomina/Colletotrichum resistance allele”), wherein each of the two or more Macrophomina/Colletotrichum resistance alleles is an allele of at least one single nucleotide polymorphism (SNP) marker selected from the SNP loci within SEQ ID NOs:8-10; and
e. selecting a strawberry plant from said population of progeny plants based on the presence of the two or more Macrophomina/Colletotrichum resistance alleles in the nucleic acid sample.

12. A method for obtaining a strawberry plant with improved resistance to Macrophomina or Colletotrichum infection, the method comprising:
a. providing an initial population of strawberry plants;
b. obtaining a nucleic acid sample from one or more individuals within said initial population;
c. detecting in each of said nucleic acid samples the presence or absence of two or more alleles conferring improved resistance to disease caused by Macrophomina and/or Colletotrichum infection (“Macrophomina/Colletotrichum resistance allele”), wherein each of the two or more Macrophomina/Colletotrichum resistance alleles is an allele of at least one single nucleotide polymorphism (SNP) marker selected from the SNP loci within SEQ ID NOs:8-10;
d. selecting a strawberry plant from said initial population based on the presence of the two or more Macrophomina/Colletotrichum resistance alleles in the nucleic acid sample; and
e. crossing the selected strawberry plant with a second strawberry plant to produce progeny plants comprising improved resistance to disease caused by Macrophomina or Colletotrichum infection.

13. The method of claim 12, wherein two strawberry plants are selected from said initial population based on the presence of the two or more Macrophomina/Colletotrichum resistance alleles in the nucleic acid sample for each of the two selected plants, and wherein the two selected plants are crossed with one another to produce progeny plants comprising improved resistance to disease caused by Macrophomina or Colletotrichum infection.

14. The method of claim 12, wherein the selected strawberry plant is homozygous for at least one of the two or more Macrophomina/Colletotrichum resistance alleles.

15. (canceled)

16. The method of claim 12, wherein the detection step comprises detecting three of the SNP loci within SEQ ID NOs:8-10.

17. The method of claim 12, wherein the detection step further comprises detecting at least one allele conferring improved resistance to disease caused by Fusarium infection (“Fusarium resistance allele”), wherein each of the at least one Fusarium resistance alleles is an allele of at least one single nucleotide polymorphism (SNP) loci within SEQ ID NOs:1-7.

18. The method of claim 17, wherein the method comprises detecting two, three, four, five, or six Fusarium resistance alleles of SEQ ID NOs:1-7.

19. The method of claim 12, wherein the detection step further comprises detecting the presence or absence of an allele conferring remontancy (“remontancy allele”), wherein the remontancy allele is a heterozygous state at a single nucleotide polymorphism (SNP) locus within SEQ ID NO:11.

20.-21. (canceled)

22. A method for obtaining a strawberry plant with improved agronomic traits, the method comprising:
a. providing an initial population of strawberry plants;
b. obtaining a nucleic acid sample from one or more individuals within said initial population;
c. detecting in each of said nucleic acid samples the presence of an allele conferring remontancy (“remontancy allele”), wherein the remontancy allele is a heterozygous state at a single nucleotide polymorphism (SNP) locus within SEQ ID NO:11;
d. selecting a strawberry plant from said population of progeny plants based on the presence of the at remontancy allele in the nucleic acid samples; and
e. crossing the selected strawberry plant with a second strawberry plant to produce progeny plants comprising improved agronomic traits.

23. The method of claim 22, wherein two strawberry plants are selected from said initial population based on the presence of the selected allele in the nucleic acid sample for each of the two selected plants, and wherein the two selected plants are crossed with one another to produce progeny plants comprising improved agronomic traits.

24. The method of claim 22, wherein the detection step further comprises detecting in each of said nucleic acid samples the presence or absence of at least one allele conferring increased berry weight (“berry weight allele”), wherein the at least one berry weight allele is an allele of at least one SNP loci within SEQ ID NOs:12-17.

25. The method of claim 24, wherein the detection step comprises detecting at least two, three, four, five, or six berry weight alleles of SEQ ID NOs:12-17.

26.-27. (canceled)


SEQUENCE LISTING (102 sequences)

SEQ ID NO: 1 - DNA, 14 bp, Fragaria sp.
tgattgtctgagaa

SEQ ID NO: 2 - DNA, 15 bp, Fragaria sp.
tgaagtatgaattaa

SEQ ID NO: 3 - DNA, 12 bp, Fragaria sp.
aagggccatgtg

SEQ ID NO: 4 - DNA, 12 bp, Fragaria sp.
caccgcctggtt

SEQ ID NO: 5 - DNA, 12 bp, Fragaria sp.
aaccatccacaa

SEQ ID NO: 6 - DNA, 11 bp, Fragaria sp.
tggcctccgag

SEQ ID NO: 7 - DNA, 12 bp, Fragaria sp.
cagctttggaat

SEQ ID NO: 8 - DNA, 14 bp, Fragaria sp.
atgcataagatgtg

SEQ ID NO: 9 - DNA, 12 bp, Fragaria sp.
actgtaacgaca

SEQ ID NO: 10 - DNA, 15 bp, Fragaria sp.
agatagctaaactca

SEQ ID NO: 11 - DNA, 12 bp, Fragaria sp.
ggatckgtgaag

SEQ ID NO: 12 - DNA, 12 bp, Fragaria sp.
ctcgccgttcta

SEQ ID NO: 13 - DNA, 12 bp, Fragaria sp.
gatcyacattct

SEQ ID NO: 14 - DNA, 12 bp, Fragaria sp.
atccttatcrac

SEQ ID NO: 15 - DNA, 12 bp, Fragaria sp.
tggtggttgttc

SEQ ID NO: 16 - DNA, 12 bp, Fragaria sp.
gccacgaaatga

SEQ ID NO: 17 - DNA, 12 bp, Fragaria sp.
aagcatcatcaa

SEQ ID NO: 18 - DNA, 401 bp, Fragaria sp.
tgttgatgatgatgaaagaagaatgagagcaacaagagtgaagagaaagcacttgtgcctccaagaacctaacattgttgaatagtaatacactttggaattgaaaagaagtgatatatactcaatagttttgtactctcaacttactgagaaggcagcagacaagttgagtaatgtaaggagactgccaacttgattgtktgagaaaagatgtggcaatggcagacagtgaggtatttgataattcttaaactgtkaaggctaagcagctcaggctgttgctctcattgtatagaatctgtgtaagtgaaagagaaaagaatttagtggattttattgttcttaattggaaccccttttttgagttgggtataagaattaaattgtttctatgtctggta

SEQ ID NO: 19 - DNA, 20 bp, Artificial Sequence
  FUS1 Forward Primer
cttactgagaaggcagcaga

SEQ ID NO: 20 - DNA, 18 bp, Artificial Sequence
  FUS1 Reverse Primer
agctgcttagccttgaca

SEQ ID NO: 21 - DNA, 14 bp, Artificial Sequence
  FUS1 T allele probe
  misc_feature (1)..(1)
  misc_feature (14)..(14)
ttctcaaacaatca

SEQ ID NO: 22 - DNA, 12 bp, Artificial Sequence
  FUS1 C allele probe
  misc_feature (1)..(1)
  misc_feature (12)..(12)
ctcagacaatca

SEQ ID NO: 23 - DNA, 400 bp, Fragaria sp.
attgcttgaccaaaatacaaaatgcaaataccttggtagagtaaggaggaagctttaggtaattagcgcatgtcattacactaggtagatcatcatctgccaattctgaagccccagtcccattaggtgctgcattattcgcagttgaagaatgctgcagcagacaaggaaacatcaacacatcaatacaccttgaagtayaattaaccattacaagtttgagggaagcaagcagccaaaaatgaggcatgaaaagaagccatagaatctcgctgtacctttctcacaatggtgagctttggattgagcactgccaaaccaccagggggaagcctaggcgcaccagtaacaaactggcaaaaggcacgctgctgctcgggcgtgaactcacccatgatct

SEQ ID NO: 24 - DNA, 21 bp, Artificial Sequence
  FUS2 Forward Primer
cccattaggtgctgcattatt

SEQ ID NO: 25 - DNA, 21 bp, Artificial Sequence
  FUS2 Reverse Primer
aggtacagcgagattctatgg

SEQ ID NO: 26 - DNA, 14 bp, Artificial Sequence
  FUS2 C allele probe
  misc_feature (1)..(1)
  misc_feature (14)..(14)
tgaagtacgaatta

SEQ ID NO: 27 - DNA, 15 bp, Artificial Sequence
  FUS2 T allele probe
  misc_feature (1)..(1)
  misc_feature (15)..(15)
tgaagtatgaattaa

SEQ ID NO: 28 - DNA, 401 bp, Fragaria sp.
ttctaaatttctggcgacgcatgtaatccatgtgaattaataagttgtgcgtagcttgattatactgcaaatggaattggggtttgagcttacacattctcgaacggcagaaatgagaggagctagaaagcttaagagagcagcagcggcaatattaggaagacttgggagatcgagtgaggagggaaggaagagaagggmcatgtgaaaaaaggcagcctttcttgttttgatgtttgtaaagcgaccacgccccgtttggctgcaatcatgtggtctactttgttataattccattccccttcactactctctaattcctcccatttctttttttgtcgtcaccctttgtcccccttccctaagtaccttgttcctctacaacattatgaaaatcacat

SEQ ID NO: 29 - DNA, 21 bp, Artificial Sequence
  FUS3 Forward Primer
cagcggcaatattaggaagac

SEQ ID NO: 30 - DNA, 19 bp, Artificial Sequence
  FUS3 Reverse Primer
cgtggtcgctttacaaaca

SEQ ID NO: 31 - DNA, 10 bp, Artificial Sequence
  FUS3 C allele probe
  misc_feature (1)..(1)
  misc_feature (10)..(10)
agggccatgt

SEQ ID NO: 32 - DNA, 11 bp, Artificial Sequence
  FUS3 A allele probe
  misc_feature (1)..(1)
  misc_feature (11)..(11)
agggacatgtg

SEQ ID NO: 33 - DNA, 401 bp, Fragaria sp.
gaacaacaacaagatgcaaccaagaaaaacagcttagttttgttgggcatcagttaagtccttgtccttctgcagccttgtacagctcttcggtacatgcatagtgtgaaagtgtggctcctttggaatggttgcaggccttcttccccgtgatgatgaccgagaaggagttcgaaccttggttgctgaagaaacaccgcmtggttttgctgctttcttgggtcttatatctgttgatatgctttcgtttattacactgagttgacaaggagtcttggacgaaaccacagacttctttttacttgaaacaaccctgcacaagaaaagtttagtaagaccaagtttcaacaagagcacaccatgtatttatcatgatggaaaattttagcaagaccaagttt

SEQ ID NO: 34 - DNA, 20 bp, Artificial Sequence
  FUS4 Forward Primer
gtgatgatgaccgagaagga

SEQ ID NO: 35 - DNA, 20 bp, Artificial Sequence
  FUS4 Reverse Primer
ttcttgtgcagggttgtttc

SEQ ID NO: 36 - DNA, 10 bp, Artificial Sequence
  FUS4 A allele probe
  misc_feature (1)..(1)
  misc_feature (10)..(10)
accgcatggt

SEQ ID NO: 37 - DNA, 10 bp, Artificial Sequence
  FUS4 C allele probe
  misc_feature (1)..(1)
  misc_feature (10)..(10)
accgcctggt

SEQ ID NO: 38 - DNA, 401 bp, Fragaria sp.
ccattgcggcattgccactaatggaactcctaaactcagagactccaaagttgagttccaaccacaatgtgtaatgaagcacccaacagcctcatgagccaaaacctctagttgggagcaccatgaaaccaccaaacccttctcagctgtctcctcgatgaacccttcggggactttagctgcttctgattctctaaccayccacaagaatttgcttttgctcctcttcaaaccccacgccagttcctccatttgctcaagtccaagttgtgcagcactgccgaatgagatgtaagcaacagagttcttcggatgtttgtttaaccatttcatgcatgcatcattgctggatttaaagaggtcaacgccataacctttgtcgtctttaagtcggttatcca

SEQ ID NO: 39 - DNA, 22 bp, Artificial Sequence
  FUS5 Forward Primer
ggactttagctgcttctgattc

SEQ ID NO: 40 - DNA, 20 bp, Artificial Sequence
  FUS5 Reverse Primer
ctgcacaacttggacttgag

SEQ ID NO: 41 - DNA, 11 bp, Artificial Sequence
  FUS5 C allele probe
  misc_feature (1)..(1)
  misc_feature (11)..(11)
accacccacaa

SEQ ID NO: 42 - DNA, 12 bp, Artificial Sequence
  FUS5 T allele probe
  misc_feature (1)..(1)
  misc_feature (12)..(12)
aaccatccacaa

SEQ ID NO: 43 - DNA, 401 bp, Fragaria sp.
gtgacggcgctgttggtggcgctgcttcctccgtcgtagtggcagcgcttgtgtcctcccaaggcctgcccggtcggaaaacacttgtggcagatggtgcactcgtggctcctcccgctaacggcggtgacagccggggtttcaaccttgacggcggaggagacggtggagtcggacttgcggtggctagccttgtggcckccgagagcctggtaagacccgaaagccttgctgcagacgacgcacttgtaagaggccgggggatgcggtgggagttgcgcgacggtggtgtcggcggcgttggtggtgccgcgggcgaggagcaagaggcagaaggccatgtactcctcctcggaaagtgatgagtcctcgaaacgcggccgcttggagcgcttacgctt

SEQ ID NO: 44 - DNA, 401 bp, Fragaria sp.
agaaacaatgtaaacagccattggcagtggctgatcatcaagctcccgagccgcttctgccgccccctcagcctccctctgcttgcacacaagagtctcccaagaccggcacatggcataagggccaacccacgaccctgcagcaaggttataacccttgcccgcctgaatcaagttatgaattgagaacaccgcagcttyggaatcaccaaacagccgcaggatcttaacatactcttgttccaatccgcaagacttgtccacaggccgcctccaagatcttcccagcctgtggaaaagcaatgcctgcgccacaagcatctggctgctccgaatcatacacccccaattcacatcactcgtaaacttggaatctccaccctctatcaaatcgaatcctc

SEQ ID NO: 45 - DNA, 20 bp, Artificial Sequence
  FUS7 Forward Primer
cccgcctgaatcaagttatg

SEQ ID NO: 46 - DNA, 21 bp, Artificial Sequence
  FUS7 Reverse Primer
gcggattggaacaagagtatg

SEQ ID NO: 47 - DNA, 11 bp, Artificial Sequence
  FUS7 C allele probe
  misc_feature (1)..(1)
  misc_feature (11)..(11)
agcttcggaat

SEQ ID NO: 48 - DNA, 11 bp, Artificial Sequence
  FUS7 T allele
  misc_feature (1)..(1)
  misc_feature (11)..(11)
agctttggaat

SEQ ID NO: 49 - DNA, 401 bp, Fragaria sp.
gagttcccacttcctggagaggaggagcgatttaaattattgaaactctacttaaacaagtatctttctggtgaagacaacacctctaaatggggcattctgaacaagaagcaacaacagaagataactatgcaggatttgtccgatgaggtgctccgagaggctgccaggaagacagatgggttctctggccgygagatmgctaaactcatggctggtgttcargcagctgtgtacgggcgcccagattgtgtactggactcccaattgtttaaggagattgtwgattacaaagttgcggagcatcaccagaggataaaactagcagctgaaggtggtgatgggatttttggcctagcttgagtgaatagaagtaggagttgaacgtctgtcatatccca

SEQ ID NO: 50 - DNA, 20 bp, Artificial Sequence
  COL 1 Forward Primer
caggaagacagatgggttct

SEQ ID NO: 51 - DNA, 20 bp, Artificial Sequence
  COL 1 Rev
tgctccgcaactttgtaatc

SEQ ID NO: 52 - DNA, 15 bp, Artificial Sequence
  COL 1 A allele prob
  misc_feature (1)..(1)
  misc_feature (15)..(15)
agatagctaaactca

SEQ ID NO: 53 - DNA, 13 bp, Artificial Sequence
  COL 1 C allele probe
  misc_feature (1)..(1)
  misc_feature (13)..(13)
agatcgctaaact

SEQ ID NO: 54 - DNA, 401 bp, Fragaria sp.
aaagygagagaaagatgccttcttgttctggaaggtcagcaaaagatgacaaggcaaatgggtcatctgatcaccgggcaaaccakgtygattcgacctcattacaatcggarcaattagctggagctgctaaccattctgctgcactagcagctgaraaactgaaccatgaagtggtcgagtggccragaatttacttgrstttgtcaaggaaggagaaagaagaygatttccttgcaatgaaaggcacgaagctgcctcagagacccaagaagagggccaagaacgttgacagaacccttcaggtagaaatattcatcacttattcttggttcatcaatttgattttggtttttgggtaattgagaaattggaacatgaattttaattttttgtggggg

SEQ ID NO: 55 - DNA, 12 bp, Fragaria sp.
acttgrstttgt

SEQ ID NO: 56 - DNA, 401 bp, Fragaria sp.
ggcccaccagcgcagaggctgcccggagcagcagcagcagccgctcttggccgtcaaagtcgaactcgagcagctcataatctcgattctcgatgaycccagtgttagtcgggtcatgcgggaggccagtttctctagtccggcagttaaagcraccatcgaacagtctcttaactcttcatcmgcggcggcggcagcgamctctaccgtcgccgcgaattcgtycccaattggattggggttccggcckgcgggaccrcccgccggtcggaacatgtatttaaatccccggctgcagggagcagcagggcaatcggggcaaaaccgagcagaggaagttaaaaaagttgctgatattttatcaagggggaagaagaggaatccggtattggtcggcgatt

SEQ ID NO: 57 - DNA, 12 bp, Fragaria sp.
agcgamctctac

SEQ ID NO: 58 - DNA, 401 bp, Fragaria sp.
ctctttctcttcataaattcacccacctcatcaaaaagcctcacatagtaaaaatccttcaaatcgatctgctcgcacaacttcttcaacacttcaaaaacctccttgtcattcttctcctcatgcacaagaatctgttttttccgcagcaatttcacatctttctcggactggatcaggcaccccattaagtatgcatamgatgtgayatgctgcaccggatcatccccaatgtgtcgctgctcgagtgcaatgaggttcttgaggagtgtttcagtgcagtgatggactaaaagaggtggcattttgaacacaccattagtaaacttgacatccaagaaactgtgtaccacggtagcacattggaatttaacccctgctctttttagcttctttacgca

SEQ ID NO: 59 - DNA, 20 bp, Artificial Sequence
  MAC 2 Forward Primer
tcacatctttctcggactgg

SEQ ID NO: 60 - DNA, 22 bp, Artificial Sequence
  MAC 2 Reverse Primer
cactgaaacactcctcaagaac

SEQ ID NO: 61 - DNA, 14 bp, Artificial Sequence
  MAC 2 A allele probe
  misc_feature (1)..(1)
  misc_feature (14)..(14)
cacatcttatgcat

SEQ ID NO: 62 - DNA, 12 bp, Artificial Sequence
  MAC 2 C allele probe
  misc_feature (1)..(1)
  misc_feature (12)..(12)
acatcgtatgca

SEQ ID NO: 63 - DNA, 401 bp, Fragaria sp.
  misc_feature (218)..(220): n is a, c, g, or t
taaaacttcctactctctgaagccagcactaggctaagatccgaaagataagactgcaagtccctgcaatcatcacaacacacaaaaaaaaaaaagcctcacttccttgctcacacaacagcaaacaaccaaaatcaacggtacattatgcgttcagcagctgaagaagcacaatgttatctctacgtatagcattactgyaacgacacacggtcacnnncacaaacaccacttawagcagcaaactcaaagcaacraaatcractacatttaggaattgagctrattattcagagtatcaatcacaactgattaaaactaattcagaacaatcgaaatcgaaagattggttacccaatttgcaaaccagtgaggggggaaatgcaccggccggcgtcact

SEQ ID NO: 64 - DNA, 19 bp, Artificial Sequence
  MAC 3 Forward Primer
tcacttccttgctcacaca

SEQ ID NO: 65 - DNA, 19 bp, Artificial Sequence
  MAC 3 Reverse Primer
ttgctttgagtttgctgct

SEQ ID NO: 66 - DNA, 11 bp, Artificial Sequence
  MAC 3 T allele probe
  misc_feature (1)..(1)
  misc_feature (11)..(11)
actgtaacgac

SEQ ID NO: 67 - DNA, 12 bp, Artificial Sequence
  MAC 3 C allele probe
  misc_feature (1)..(1)
  misc_feature (12)..(12)
actgcaacgaca

SEQ ID NO: 68 - DNA, 601 bp, Fragaria sp.
gaaatttcgactacatgtcatatatgattcagcaaaatcacaaagttaaagaattcagcatccaaaattcaaacttgcaaacaaatcaaccaacacaattacatataattcccgggaaatttgggtcctgagatgtaatacctgggataagacccttctgaatcttctggacctgtttgcagtttaccccagaaagaacgagaaagtgagacggttggccttaactacctcattcagttttaccgttagagaccaaggttaggtggactttgaagtttgctgcggccttcttctgggatckgtgaagctgaacaagtctgtctgattggtcaaaactgattggtgctacagcaacagctaaaacagtgaaactcaaacagctgccgtactcagaatgttgtgttgtgcggtgtacgctgtcactttggaccttcaataattgagtgtactaatctacaaattttgtaacgctccctgatcttaataactgtggtttaaatataccagatcatctgcagttcttttgcatgtgatttggtgcataaagccggtggttaagcaattgaacgcaggggatttcaatagtttatgaaattaatgg

SEQ ID NO: 69 - DNA, 18 bp, Artificial Sequence
  ROM 1 Forward Primer
taggtggactttgaagtt

SEQ ID NO: 70 - DNA, 19 bp, Artificial Sequence
  ROM 1 Reverse Primer
caccaatcagttttgacca

SEQ ID NO: 71 - DNA, 14 bp, Artificial Sequence
  ROM 1 G allele probe
  misc_feature (1)..(1)
  misc_feature (14)..(14)
gggatcggtgaagc

SEQ ID NO: 72 - DNA, 14 bp, Artificial Sequence
  ROM 1 T allele probe
  1misc_feature (1)..(1)
  1misc_feature (14)..(14)
gggatctgtgaagc

SEQ ID NO: 73 - DNA, 401 bp, Fragaria sp.
acaggagtgcatgtggcgctgaagtttgttataagctcsagccagatcctcggcctcttgacaaacgctgccggtgaagtcaaaacacatgtygcwccggagacaatggtcaacaacaagaacatgagrccgcaatcatggtactgaggcagccacgrcacgatcacactgttcgggtgaaggtcgtaggatttcctcgcmgttctaacgttgtgaactgccgagccggmggtgacaagyaccggctttggaatcccggtagtacctgasgtgtactgaatcaaatatarctcgtcagctttgcagccctcgtaggacgaagggctgaactgtaaattcgagtttctagctccggttttgatatcatcggtggatatccagtggagattctccaacaagtg

SEQ ID NO: 74 - DNA, 20 bp, Artificial Sequence
  BW 1 Forward Primer
gggtgaaggtcgtaggattt

SEQ ID NO: 75 - DNA, 20 bp, Artificial Sequence
  BW 1 Reverse Primer
ggtactaccgggattccaaa

SEQ ID NO: 76 - DNA, 11 bp, Artificial Sequence
  BW 1 A allele probe
  misc_feature (1)..(1)
  misc_feature (11)..(11)
tcgcagtccta

SEQ ID NO: 77 - DNA, 11 bp, Artificial Sequence
  BW 1 C allele probe
  misc_feature (1)..(1)
  misc_feature (11)..(11)
tcgccgtccta

SEQ ID NO: 78 - DNA, 401 bp, Fragaria sp.
tgaactcttctgagaacaatcctgccaactgctgccatagctcaacttttgaaggctatcgaaaatatacatccgtcctttcggtatatttgagctgctagccaattcctttmgagattctctcrgtcaaatccgaaagaagagcatacttgttrctctcgtaacatgcagtcgtaaacgcttgcaatgtcacacgatcyrcattctgatcctyatcractaatttgtgaaagaacaatgctgctattcccactttcttctcacaacaaagcttcctgaccaatgtattaaccgtgcgratccagtacttcttttcaagcctatctaagatgaccatagcagtggcatagtcatctttttggcaatacttgtgagttaatgtcaacctagttacttcacag

SEQ ID NO: 79 - DNA, 23 bp, Artificial Sequence
  BW 2 Forward Primer
tcaaatccgaaagaagagcatac

SEQ ID NO: 80 - DNA, 22 bp, Artificial Sequence
  BW 2 Reverse Primer
gcagcattgttctttcacaaat

SEQ ID NO: 81 - DNA, 11 bp, Artificial Sequence
  BW 2 A allele probe
  misc_feature (1)..(1)
  misc_feature (11)..(11)
atgtggatcgt

SEQ ID NO: 82 - DNA, 11 bp, Artificial Sequence
  BW 2 G allele probe
  misc_feature (1)..(1)
  misc_feature (11)..(11)
atgcggatcgt

SEQ ID NO: 83 - DNA, 401 bp, Fragaria sp.
gaacaatcctgccaactgctgccatagctcaacttttgaaggctatcgaaaatatacatccgtcctttcggtatatttgagctgctagccaattcctttmgagattctctcrgtcaaatccgaaagaagagcatacttgttrctctcgtaacatgcagtcgtaaacgcttgcaatgtcacacgatcyrcattctgatcctyatcractaatttgtgaaagaacaatgctgctattcccactttcttctcacaacaaagcttcctgaccaatgtattaaccgtgcgratccagtacttcttttcaagcctatctaagatgaccatagcagtggcatagtcatctttttggcaatacttgtgagttaatgtcaacctagttacttcacagggcgacagcccct

SEQ ID NO: 84 - DNA, 23 bp, Artificial Sequence
  BW 3 Forward Primer
tcaaatccgaaagaagagcatac

SEQ ID NO: 85 - DNA, 22 bp, Artificial Sequence
  BW 3 Reverse Primer
gcagcattgttctttcacaaat

SEQ ID NO: 86 - DNA, 14 bp, Artificial Sequence
  BW 3 T allele probe
  misc_feature (1)..(1)
  misc_feature (14)..(14)
tgataaggatcaga

SEQ ID NO: 87 - DNA, 12 bp, Artificial Sequence
  BW 3 C allele probe
  misc_feature (1)..(1)
  misc_feature (12)..(12)
tgatgaggatca

SEQ ID NO: 88 - DNA, 401 bp, Fragaria sp.
agcattgtgtggtcacatggcttgtcactctggtgacaaggagaggaagcttgaagagattgaggagttttccgagactagtgagaagcataagctagtgatggatagtcagtctgatactgagacctcatcagctcmaacaaagcagaggagggtgtcaaagagattgagggasagcwgcaactttgaagttcatggtgrttgttcatctggttctggagttgagcaagagcagcaggaagtggctattagtttgatgatgttgtctcgtgattcgggttataaagggggtttgaattcgtttggtgagtcttcagataacaattcagtgattttggagggtaagtcatcttctattgatatgagaattagtactaagagaggtctgagttatggtgcta

SEQ ID NO: 89 - DNA, 20 bp, Artificial Sequence
  BW 4 Forward Primer
gaggagggtgtcaaagagat

SEQ ID NO: 90 - DNA, 23 bp, Artificial Sequence
  BW 4 Reverse Primer
cacgagacaacatcatcaaacta

SEQ ID NO: 91 - DNA, 11 bp, Artificial Sequence
  BW 4 G allele probe
  misc_feature (1)..(1)
  misc_feature (11)..(11)
caaccaccatg

SEQ ID NO: 92 - DNA, 12 bp, Artificial Sequence
  BW 4 A allele probe
  misc_feature (1)..(1)
  misc_feature (12)..(12)
caatcaccatga

SEQ ID NO: 93 - DNA, 401 bp, Fragaria sp.
acgcaacctgctgcagtgcatatatcacctccgccaccgaaaartactgctgcatatgaagcaccggtgtccaattgcacctcctctgctgaacacacccgatcaccatgtcatactcactcggctctcccaccgcccgcaaatggtggcacagcgagtcaataatcgcattggccgccgcaaactcaccccgcwgccacraaatgaacccgtcacgttcatcsgggaaccactgccgcggcwgctggtggatctctccgccagatacagcggctccggcgacactaggatactgcattttgtccgataaaaccacattccctgatggcattgtcattagaaaacccactacacaaaattccctaacagcaacaattaatcattccctcactatatctcct

SEQ ID NO: 94 - DNA, 20 bp, Artificial Sequence
  BW 5 Forward Primer
ggcacagcgagtcaataatc

SEQ ID NO: 95 - DNA, 18 bp, Artificial Sequence
  BW 5 Reverse Primer
cagtggttcccggatgaa

SEQ ID NO: 96 - DNA, 12 bp, Artificial Sequence
  BW 5 G allele probe
  misc_feature (1)..(1)
  misc_feature (12)..(12)
agccacgaaatg

SEQ ID NO: 97 - DNA, 13 bp, Artificial Sequence
  BW 5 A allele probe
  misc_feature (1)..(1)
  misc_feature (13)..(13)
agccacaaaatga

SEQ ID NO: 98 - DNA, 401 bp, Fragaria sp.
agccaatggactcaccaaagaacaaggccgcaagtacagcgactgtcaaaggttgtgaatcaattatgacctgtaatgaatgccagggtcaaaagggtgtagacatgaaatgtgatagttgttaggtaaagtaactcacgctgcccagaccagcagacgtyctctgcaaaccttgagcaagaaatccttggaaacaagcakcatcaacgaggccgaagagcgcaatagaaagccaagcggtgaggccagaaggaaasgggcggcctctgtaggcagcgaaggcgatgaggaggaagccagcaggaatcagacggaaggctgagatgaagaaagggccggtctttggcagaatgcccttcatggccaccatagccgtaccccaaaagaagaagggagagacc

SEQ ID NO: 99 - DNA, 26 bp, Artificial Sequence
  BW 6 Forward Primer
tgaaatgtgatagttggtaggtaaag

SEQ ID NO: 100 - DNA, 20 bp, Artificial Sequence
  BW 6 Reverse Primer
caccgcttggctttctattg

SEQ ID NO: 101 - DNA, 12 bp, Artificial Sequence
  BW 6 G allele probe
  misc_feature (1)..(1)
  misc_feature (12)..(12)
aagcagcatcaa

SEQ ID NO: 102 - DNA, 14 bp, Artificial Sequence
  BW 6 T allele probe
  misc_feature (1)..(1)
  misc_feature (14)..(14)
aagcatcatcaacg